(19) United States
(12) Patent Application Publication
(10) Pub. No.: US 2005/0277697 A1
(43) Pub. Date: Dec. 15, 2005

(54) Method for treatment of the common cold, flu, and virus symptoms

(76) Inventor: Allan Anthony Wasson, Indianapolis, IN (US)

Correspondence Address: Allan Anthony Wasson, 11328 East Saint Joseph Street, Indianapolis, IN, 46229, US

(21) Appl. No.: 10/868,531

(22) Filed: Jun. 15, 2004

(51) Int. Cl. A61K 31/137

(52) U.S. Cl. 514/649

(57) ABSTRACT

This invention relates to the treatment of a person exhibiting the effects of a flu-like or viral infection. The method includes treating the person with an amphetamine, amphetamine derivative, or a salt thereof in an amount effective to alleviate the adverse symptoms resulting from the flu-like or viral infection.

### BACKGROUND OF THE INVENTION

The present invention relates to the treatment of flu-like infections, viral infections, and symptoms thereof.

People, particularly in the winter time, suffer from a wide variety of viral infections. These infections can range from relatively mild cases that cause minor discomfort and little disruption of normal activity to more severe cases which can be life threatening. The symptoms, depending upon the virus, can vary widely. Typically, the onset is very rapid (usually within one to three days). The illness typically begins with nasal or throat discomfort, followed by sneezing, rhinorrhea, myalgia, and general malaise. In addition, a hacking cough, runny nose, and watery eyes may accompany the illness. In more extreme cases, persistent bronchitis can develop in people that exhibit chronic respiratory tract diseases. Currently, not all viral infections can be adequately treated. For example, the common cold currently has no known definitive cure.

It is common to treat the above-mentioned symptoms with a variety of treatment regimes and palliative care. Selected adrenergic drugs have been used to treat individuals suffering from one or more of the above symptoms. However, many adrenergeic agents are addictive. Consequently, it is believed that many of these agents are not considered for treating viral infections.

In light of the above-described problems, there is a continuing need to provide new and improved treatments and treatment methods for flu and virus-like infections and their symptoms.

### SUMMARY OF THE INVENTION

The present invention relates to methods for treating viral infections, flu-like infections, and symptoms thereof. Various aspects of the invention are novel, nonobvious, and provide various advantages. While the actual nature of the invention covered herein can only be determined with reference to the claims appended hereto, certain forms and features, which are characteristic of the preferred embodiments disclosed herein, are described briefly as follows.

In one form, the present invention provides a method of treating people who suffer from flu or viral infections or are exhibiting adverse effects or symptoms of the infections. The method comprises administering to the person a pharmaceutically-acceptable salt of an amphetamine or a derivative thereof in a unit dosage form in an amount sufficient to alleviate one or more of the adverse effects. The amphetamine salt or derivative thereof can be selected from a wide variety of pharmaceutically-acceptable salts and derivatives. In a preferred embodiment, the amphetamine salt is a salt of dextroamphetame and/or amphetamine. In other embodiments, the salt can be an aspartate salt, a sulfate salt, or a saccharate salt of the amphetamine or a derivative thereof.

In another form, the present invention provides a method of treating a person exhibiting one or more symptoms including runny nose, watery eyes, sneezing, persistent coughing, and/or viral-induced malaise. The method comprises administering to the person an effective amount of a pharmaceutically-acceptable salt of an amphetamine or a derivative thereof.

Further objects, features, aspects, forms, advantages and benefits of the present invention shall become apparent from the description contained herein.

### DETAILED DESCRIPTION OF THE PREFERRED EMBODIMENT

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described compositions and treatment methods, and any further applications of the principles of the invention as described herein, are contemplated as would normally occur to one skilled in the art to which the invention relates.

In one form, the present invention provides a method of treating a person who is suffering from one or more symptoms related to a viral or a flu-like infection. Such symptoms include, without limitation, inflammation and discomfort within the nasal passages or throat, laryngitis, tracheitis, nasal secretions, watery eyes, a cough or bronchitis, tracheobroncheal involvement, and even pneumonia. These symptoms can last several days depending upon the severity and the type of infection. In the past, nasal decongestants, analgesics, and antipyretics have been commonly used. However, their effects can be temporary and possibly of insufficient magnitude. Furthermore, antibiotics are not effective against viruses and are typically not recommended unless specific complications arise.

The present invention provides a treatment for a wide variety of symptoms including those listed above. The treatment comprises administering to the person that exhibits these symptoms or viral or flu-like effects a pharmaceutically-acceptable salt of an amphetamine derivative in unit dosage form. In preferred forms, the present invention provides a method of treating individuals suffering from viral allergies and flu-like symptoms. The treatment method allows affected individuals to function effectively even under demanding physical or mental activities. Consequently, the treatment can allow the affected individuals to continue their normal activities including working in demanding situations and maintaining their high cognitive state without experiencing the drowsiness normally associated with antihistamines and/or decongestants.

The pharmaceutically-acceptable salt can be provided or administered to the person in a wide variety of routes. The pharmaceutical compositions disclosed herein may be orally administered, for example, with an inert diluent or with an assimilable edible carrier; enclosed in a hard- or soft-shell gelatin capsule; compressed into tablets; or incorporated directly with the food of the diet. For oral therapeutic administration, the active compounds may be incorporated with excipients and used in the form of ingestable tablets, capsules, elixirs, suspensions, syrups, wafers, and the like. Such compositions and preparations should contain at least 0.1% of active compound. The percentage of the compositions and preparations may, of course, be varied and may conveniently be between about 1 to about 60% of the weight of the unit. The amount of active compounds in such therapeutically-useful compositions is such that a suitable dosage will be obtained.

When the pharmaceutical formulation is provided as a tablet or pill, the tablets, pills, capsules, and the like may also contain the following: a binder, such as gum tragacanth, acacia, cornstarch, or gelatin; excipients, such as dicalcium phosphate; a disintegrating agent, such as corn starch, potato starch, alginic acid, and the like; a lubricant, such as magnesium stearate; and a sweetening agent, such as sucrose, lactose, or saccharin or a flavoring agent, such as peppermint, oil of wintergreen, or cherry flavoring, may be added. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparations and formulations.

In preferred forms, the active ingredient includes an amphetamine, an amphetamine derivative, and/or a salt thereof. A particularly preferred composition does not exhibit addictive behavior in humans. When the amphetamine or amphetamine derivative is provided as a salt, the salt can be provided as a pharmaceutically-acceptable acid addition salt, which includes salts derived from inorganic acids such as hydrochloric, nitric, phosphoric, sulfuric, hydrobromic, hydriodic, phosphoric, and the like, as well as the salts derived from organic acids, such as aliphatic mono- and di-carboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, aspartate, succinate, saccharate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, maleate, tartrate, methanesulfonate, and the like. Also contemplated are salts of amino acids such as arginate and the like and gluconate, galacturonate (see, for example, Berge S. M., et al., “Pharmaceutical Salts,” J. of Pharmaceutical Science, 1977; 66:1-19).

The effective dosage of active ingredient employed may vary depending on the particular compound employed, the mode of administration, and the severity of the condition being treated. However, in general, active ingredients can be administered at a daily dosage as medically prudent and effective. For example, a daily dosage can be between 0.01 to about 0.5 mg/kg of animal body weight per day, preferably given in divided doses two to four times a day or in sustained-release form. Dosage forms suitable for internal use comprise the active compound in intimate admixture with a solid or liquid pharmaceutically-acceptable carrier. This dosage regimen may be adjusted to provide the optimal therapeutic response. For example, several divided doses may be administered daily or the dose may be proportionally reduced as indicated by the exigencies of the therapeutic situation.

The active ingredient may be provided in a variety of formulations. In preferred embodiments, the formulation can be provided as a controlled-release system or formulation. Such approaches may involve various types of controlled-release systems, ranging from one which may for example be based on a polymer which simply provides a delayed release of the complex with time, through a system which is resistant to dissociation under acidic conditions, for example by the use of buffering, to a system which is biased towards release under conditions that prevail in the small intestine, for example a pH sensitive system which is stabilized towards a pH of 1 to 3 such as prevails in the stomach but not one of 7 to 9 such as prevails in the small intestine.

The active ingredients for the present invention are commercially available. For example, a variety of commercial formulations comprise either amphetamine, dextroamphetamine and/or pharmaceutically-acceptable salts thereof. One particular commercially-available formulation is sold under the trade name ADDERALL sold by Shire may be suitable for use in the present invention.

### EXAMPLE

It has been unexpectedly determined that a person who was regularly taking ADDERALL prescribed by a doctor for ADD (Attention Deficit Disorder) exhibited symptoms related to a viral infection that included headache, runny nose, coughing. This person was associated with a variety of other individuals who also exhibited similar symptoms. The person missed some dosages of the prescribed ADDERALL. Upon ingesting the recommended dosage of the ADDERALL, the person, within one hour, experienced complete relief of the runny nose, cough, and sinus and chest congestion. However, the symptoms would reappear upon skipping the prescribed dosage of ADDERALL. Therefore, it has been observed that the ingestion of an amphetamine, derivatives of amphetamines, and their salts can provide essentially immediate relief for the symptoms generated from flu-like or viral infections. This result has been totally unexpected based upon labeled uses of the medication.

1. A method of treating a person exhibiting adverse effects of a flu-like or viral infection, said method comprising:
administering to said person an amphetamine, amphetamine derivative, or salt thereof in a unit dosage form in an amount sufficient to alleviate one or more of the adverse effects.

2. The method of claim 1, wherein said amphetamine derivative or salt thereof is selected from the group consisting of dextroamphetamine, amphetamine, dextroamphetamine saccharate, dextroamphetamine sulfate, amphetamine aspartate, and mixtures thereof.

3. The method of claim 1, wherein said administering comprises administering to said person the amphetamine, amphetamine derivative, or salt thereof in a table form.

4. The method of claim 1, wherein the viral infection is a result of a virus of unknown etiology.

5. A method of treating a person exhibiting adverse effects of an air-born allergen, said method comprising administering to said person an amphetamine, amphetamine derivative, or salt thereof in a unit dosage form in an amount sufficient to alleviate one or more of the adverse effects.
